(19) United States
(12) Patent Application Publication
(10) Pub. No.: US 2016/0002679 A1
(43) Pub. Date: Jan. 7, 2016

(54) METHOD OF INCREASING LIPID ACCUMULATION IN METSCHNIKOWIA PULCHERRIMA CELLS

(71) Applicant: UNIVERSITY OF BATH, Bath (GB)

(72) Inventors: Christopher CHUCK, Bristol (GB); Fabio SANTOMAURO, Bath (GB); Roderick SCOTT, Bath (GB)

(73) Assignee: University of Bath, Bath (GB)

(21) Appl. No.: 14/654,372

(22) PCT Filed: Feb. 3, 2014
(86) PCT No.: PCT/GB2014/050288

§ 371 Date: Jun. 19, 2015

(30) Foreign Application Priority Data
Feb. 7, 2013 (GB) 1302197.7

(51) Int. Cl. C12P 7/64; A23K 1/16; C10L 1/02; C12N 1/16; C11C 3/00

(52) U.S. Cl. CPC C12P 7/64; C12N 1/16; C12P 7/6409; C12P 7/6445; C11C 3/00; C10L 1/02; A23K 1/164; C10L 2200/0469

(57) ABSTRACT

The invention relates to a method of increasing lipid accumulation in Metschnikowia pulcherrima (Candida pulcherrima) cells. In particular, the invention relates to a method of obtaining oil from yeast pulcherimma cells. The invention further relates to an oil and the use of pulcherimma cells for production of oleaginous biomass.

### BACKGROUND TO THE INVENTION

Currently the main source of biofuels globally is from sugar derived bioethanol or biodiesel produced from lipids. Most biodiesel is produced from palm, rapeseed or soybean oils. However, these crops compete for land under cultivation for the production of food and consequently have a negative public image, a negative environmental impact and can only be produced in comparatively small amounts.

An alternative to using higher plants for lipid production is to cultivate oleaginous microorganisms such as microalgae, bacteria, fungi and yeasts. While species from all of these taxa have the potential to produce lipids, only phototrophic microalgae have been significantly investigated to date. Although algae have great theoretical potential as a fuel source, substantial technical hurdles currently prevent their cost effective exploitation. These include the scarcity of suitable land area, given the current levels of productivity, the lack of adequate photosynthetically active radiation and the prohibitive costs of delivering supplementary light, controlling the temperature and critically, protecting against invasive non-lipid producing species.

Heterotrophic organisms such as yeasts, are a highly credible alternative to microalgae for the production of biofuel feedstocks, especially in Northern Europe.

Oleaginous yeast species are highly productive on a per cell basis, with lipid yields of up to 65% of the dry weight under suitable conditions and can grow to high densities with biomass yields of 10-100 gl⁻¹ being reported over 3-7 days.

The most common yeasts examined for biofuel production are Rhodotorula glutinis, Yarrowia lipolytica or Lipomyces starkeyi. In comparison, microalgae achieve only 0.15-0.25 gl⁻¹ per day in open pond systems. Recently the economic production of C₅ and C₆ sugars from waste cellulosic materials has become plausible, and plant sugars derived from lignocellulose are now a feasible source of renewable material. Unlike microalgae, yeast cultivation does not require light, which both reduces input costs and enables continuous production.

Importantly, yeast fermentation does not require agricultural land, avoiding displacement of food production. Additional inputs such as phosphorous and nitrogen are easily obtainable from waste streams such as waste water, again reducing input costs.

However, a key issue with the cultivation of heterotrophic organisms is maintaining an axenic population. Bacteria from the surrounding environment, as well as those contaminating the waste streams, will outcompete most types of oleaginous yeast. As such, to insure the strict sterile conditions required for yeast production of triglycerides, severe pre-treatment of the feedstock must first be undertaken as well as careful enclosure of the fermentation broth. This significantly increases the cost of production and has large ramifications for producing lipid biofuels on an industrial scale.

Metschnikowia pulcherrima is considered a non-oleaginous yeast and is not considered to accumulate lipids to any significant degree (Chatzifragkou et al. Energy, 2011, 36, 1097-1108).

This yeast is most commonly isolated from fruits and flowers—especially grape skins. The majority of research has therefore focussed on the effect that M. pulcherrima has on the taste and aroma of wines. As a non-Saccharomyces yeast involved in the first stages of wine fermentation, M. pulcherrima is able to endure particularly stressful conditions such as a high acidic environment due to the high levels of tartaric and malic acid, and a high osmotic pressure due to the high content of sugar. Recent studies have shown its ability to produce a wide range of enzymes, demonstrating its metabolic plasticity and possible industrial applications in oenology.

M. pulcherrima is also able to excrete pulcherrimin, which has a high affinity to iron, forming soluble metal-organic frameworks. M. pulcherrima is both acidophilic, mesophillic and can secrete anti-microbial compounds into the culture medium. Antimicrobial properties of pulcherrimin have been demonstrated, leading to M. pulcherrima being used as a biofungicide in post-harvest disease control.

During its life cycle, M. pulcherrima undergoes sporulation and during the transition it passes through a peculiar phenotypic stage, termed “pulcherrima” cells. These cells have not previously been reported in densities of more than 0.1%. During progression of the cycle, these oval-shaped vegetative pulcherrima cells form spores. Other species of yeast produce similar cells.

It should be noted that M. pulcherrima, is also reported in literature as Candida pulcherrima (anamorph), Saccharomyces pulcherrimus, Rhodotorula pulcherrima, Torula pulcherrima, Cryptococcus castellanii and Torulopsis pulcherrima.

It would be desirable to provide an improved method of increasing accumulation of lipid in pulcherimma cells.

### SUMMARY OF THE INVENTION

The present invention provides a method in which sporulation is blocked to trigger vegetative pulcherimma cells to accumulate oil and form oil-rich cells at high cell density. The method provides increased production of pulcherrima cells and an accumulation of lipid in the cells, which may be extracted as an oil composition useful as a biofuel.

The yeast is cultured under stressed conditions to trigger pulcherimma cells to accumulate lipid. Oil from these cells can therefore be obtained using inexpensive non-sterile conditions for biofuel production.

One aspect of the invention provides a method of increasing lipid accumulation in pulcherrima cells as claimed in claim 1.

Another aspect of the invention provides an oil as claimed in claim 38.

Another aspect of the invention provides a fuel, fuel substitute, base for cosmetics, animal feed or plastic as claimed in claim 42.

Another aspect of the invention provides a use pulcherrima cells as claimed in claim 43.

Yet another aspect of the invention provides a yeast culture of as claimed in claim 44.

Another aspect of the invention provides a pulcherrima cell as claimed in claim 47.

The invention provides a method of increasing lipid accumulation in pulcherrima cells by culturing a yeast in a culture medium under conditions suitable for promoting production of pulcherrima cells and inhibiting sporulation.

Preferably, the method comprises a first step of culturing the yeast under conditions suitable for promoting production of vegetative pulcherimma cells,

and a second step of culturing the yeast under conditions suitable for inhibiting sporulation.

In a preferred embodiment, the first step comprises providing the yeast with at least one nitrogen and/or sulphur source, and at least one carbon source.

Preferably, the nitrogen and/or sulphur source is provided in the culture medium in a limiting concentration to induce starvation of the yeast.

The at least one nitrogen source may be provided at the limiting concentration of about 0.15-1.4 g/L.

Preferably, the at least one nitrogen source may be provided at the limiting concentration of about 0.2 g/L.

In one embodiment, the least one sulphur source is provided in a limiting concentration of about 0.04 g/L or lower.

Preferably, the amount of available carbon is 12 g/L or higher.

The ratio of carbon to nitrogen may be about 60:1.

The ratio of carbon to sulphur may be about 300:1

The first step may comprise maintaining a temperature of about 10-28° C.

Preferably, the first step may comprise maintaining a temperature of about 20-25° C.

The first step may comprise maintaining the pH at about 4 to 6.

Preferably, the first step may comprise maintaining the pH at 4-4.5.

Preferably, the second step comprises lowering the temperature.

More preferably, the step of lowering the temperature comprises adjusting the temperature to below about 20° C.

Preferably, the step of lowering the temperature comprises adjusting the temperature to below about 10-20° C.

More preferably, the temperature is lowered to between about 12° C. to 20° C.

Preferably, the second step comprises adjusting the pH to between about 2 to 4.

The second step may comprises adjusting the pH to between about 2 to 3.5.

In one embodiment, the first step comprises providing the yeast with biotin.

Advantageously, the second step is performed following depletion of nitrogen and/or sulphur from the culture medium to a level at which normal growth of the yeast is not sustained.

In one embodiment, the level of nitrogen at which normal growth of the yeast is not sustained is about 0.106 g/L.

Another aspect of the invention provides an oil comprising a lipid profile comprising 0-50% sterol, and 50-100% triglycerides.

Advantageously, the oil has a dynamic viscosity of about 0.58 Pa measured at 40° C.

Advantageously, the oil has an energy density of 27.33 MJ/kg.

The yeast may be selected from: Metschnikowia pulcherrima, Metschnikowia fructicola, Metschnikowia reukaufi, Candida albicans, Chlamydozyma zygote, Metschnikowia vanudenii, Metschnikowia lachancei, Hansenula saturnus and Debaryomyces dekkeri.

Advantageously, the method comprises the step of obtaining oleaginous biomass from the culture medium.

Advantageously, the oleaginous biomass comprises lipid at about 40% of total dry weight.

Advantageously, the lipid comprises sterols, triglycerides and/or free fatty acids.

The triglycerides may comprise palmitic acid, palmitoleic acid, stearic acid, oleic acid and/or linoleic acid.

Preferably, the at least one carbon source is selected from glycerol, lignocellulose, sugar, polysaccharides, oligosaccharide, waste water, waste foods, agricultural waste or energy crops.

Preferably, the at least one carbon source comprises glucose.

The at least one carbon source may comprise glycerol added to the culture medium at a concentration of 3 to 5 wt %.

In one embodiment, the at least one nitrogen source comprises ammonium salts.

The culture medium may comprise nutrients selected from salts of manganese, zinc, sodium potassium, calcium, magnesium and/or iron.

Advantageously, the culture medium is unsterilized culture medium.

Advantageously, the yeast is cultured in a substantially open reactor.

The reactor may comprise an open raceway pond.

The method may further comprise the step of dewatering the oleaginous biomass.

Advantageously, the step of dewatering the oleaginous biomass comprises a self-flocculation step.

The step of dewatering the oleaginous biomass may comprise a precipitation step.

The method may further comprise the step of extracting lipid from the oleaginous biomass.

Preferably, the step of extracting lipid from the oleaginous biomass is by solvent or microwave extraction.

The step of extracting lipid from the oleaginous biomass may be performed at between 3-15 days.

The method may comprise at least one step of further chemical upgrading.

The chemical upgrading may be to produce a fuel, fuel substitute, base for cosmetics, plastic or animal feed.

Advantageously, the oleaginous biomass further comprises co-products.

Advantageously, the co-products comprise pulcherrimin pigment, pulcherriminic acid, animal feed, ethyl caprylate and/or ethyl acetate.

Advantageously, the co-products comprise pulcherrimin pigment, pulcherriminic acid, animal feed, ethyl caprylate, acetoin, isoamyl alcohol, 2,3 butanediol, acetic acid, acetaldehyde, n-propanol, 1,2-methyl-1-propanol, 2,3-butanediol, 2-phenylethanol, geranyl acetate, geranyl alcohol, ethyl acetate, ethyl hexanoate and/or ethyl decanote.

Another aspect of the invention provides an oil comprising a lipid profile comprising 0-50% sterol, 50-100% glyceride and 0-10% free fatty acids.

The oil may have a dynamic viscosity of about 0.58 cP measured at 40° C.

The oil may have an energy density of 27.33 MJ/kg.

Advantageously, the oil is a bio-oil.

Another aspect of the invention provides a fuel, fuel substitute, base for cosmetics, animal feed or plastic comprising the oil.

Yet another aspect of the invention provides the use of pulcherrima cells for production of oleaginous biomass.

In one embodiment the use of pulcherrima cells is for production of oleaginous biomass to form a biofuel.

Yet another aspect of the invention comprises a yeast culture comprising pulcherimma cells at greater than about 0.1%(w/v).

The yeast culture may comprise pulcherimma cells at greater than about 20% (w/v).

The yeast culture comprising pulcherimma cells at greater than about 40%(w/v).

More preferably, the culture provides pulcherimma cells at about 40%-50%(w/v).

The yeast culture may be a culture of Metschnikowia pulcherrima, Metschnikowia fructicola, Metschnikowia reukaufi, Candida albicans, Chlamydozyma zygote, Metschnikowia vanudenii, Metschnikowia lachancei, Hansenula saturnus or Debaryomyces dekkeri.

Yet another aspect of the invention provides a pulcherimma cell comprising lipid at about 25-80% (w/v).

Preferably, the pulcherimma cell comprises lipid at about 40-70% (w/v).

The pulcherimma cell may be a cell from Metschnikowia pulcherrima, Metschnikowia fructicola, Metschnikowia reukaufi, Candida albicans, Chlamydozyma zygote, Metschnikowia vanudenii, Metschnikowia lachancei, Hansenula saturnus or Debaryomyces dekkeri.

### BRIEF DESCRIPTION OF THE FIGURES

FIG. 1 shows an example of the effect of glycerol concentration on the growth of M. pulcherrima.

FIG. 2 shows an example of the Effect of glycerol concentration on the total lipid content, analysed by green fluorescence, of M. pulcherrima

FIG. 3 shows an example of the effect of nitrogen source on the a) growth of the culture and b) total lipid content, analysed by green fluorescence, for M. pulcherrima

FIG. 4 shows an example of the effect of mineral depletion on the growth, after 15 days, of M. pulcherrima

FIG. 5 shows an example of the effect of mineral depletion on the total lipid content, analysed by green fluorescence, of M. pulcherrima

FIG. 6 shows an example of the effect of reducing the available sulphur on the a) growth of the culture and b) total lipid content, analysed by green fluorescence, for M. pulcherrima

FIG. 7 shows an example of the. effect of pH on the a) growth of the culture and b) total lipid content, analysed by green fluorescence, for M. pulcherrima

FIG. 8 shows an example of the effect of temperature on the a) growth of the culture and b) total lipid content, analysed by green fluorescence, for M. pulcherrima

FIG. 9 shows an example of the. Optical density of the culture after 15 days cultured on alternative sugar sources, where GLU=glucose, XYL=xylose, GLY=glycerol, ARA=arabinose, CELL=celliobiose LAC=lactose, SUC=sucrose

FIG. 10 shows an example of the. Growth of the culture on waste water and glycerol, or in a minimal media containing K and Na phosphate, ammonia and glycerol.

FIG. 11 shows an example of the temperature, pH and growth curve for the M. pulcherrima culture, cultivated in an open air stirred tank reactor.

FIG. 12a shows a graphical representation of vegetative pulcherimma cells.

FIG. 12b shows a representation of oil-rich pulcherimma cells.

FIG. 13 shows an example of the effect of initial pH on lipid accumulation.

### MATERIALS AND METHODS

M. pulcherrima was first analysed for its ability to accumulate lipids by triggering sporulation and holding the cells in this state, to do this a range of conditions were tested.

The ability of the yeast to metabolise mixtures of sugars was further examined, enabling production from the most heterogeneous of biomass sources and finally the effectiveness of producing lipids inexpensively was examined by culturing in a 500 L, open air, tank reactor.

### Materials

All chemicals were purchased from Sigma Aldrich unless otherwise stated and used without purification prior to use.

### Culture Conditions on a Laboratory Scale

Initially pre-inoculation cultures were grown from a single colony taken from a YMD agar plate, dissolved in 10 ml YMD (yeast extract 10 g/L; malt extract 20 g/L; glucose 20 g/L). The cultures were maintained at 25° C. with an agitation rate of 180 rpm;

The media used throughout the initial experiments was made up of: KH₂PO₄ 7 g/L; Na₂HPO₄ 2.5 g/L, MgSO₄.7H₂O 1.5 g/L; CaCl₂.2H₂O 0.15 g/L; ZnSO₄.7H₂O 0.02 g/L; MnSO4.H₂O 0.06 g/L, FeCl₃ 0.15 g/L; (NH₄)₂SO₄ 0.5 g/L and yeast extract 1 g/L.

All media was autoclaved for 2 hours at 120° C. prior to use. To examine the effect of temperature and nutrients on the lipid profile these conditions were then varied; in all experiments 3 replicates were used. All experiments were undertaken in 10 ml cultures inoculated into 50 ml falcon tubes.

The effect of glycerol was examined, where concentrations between 1% and 25% (w/v) were used. 30 g/L of glycerol was then used for all subsequent experiments.

To examine the effect of temperature on the lipid production, the temperature was held at 25° C. and then switched to either 20° C. or 15° C. after 3 days.

This method was also used to examine the effect of removing the micronutrients in the sample.

Three alternative nitrogen sources: NH₄Cl, NH₄NO₃ and Ca(NO₃)₂, were also examined by replacing (NH₄)₂SO₄ in the original media. These reagents were added in precise concentrations to achieve a consistent nitrogen quantity (N=0.106 g/L). Glycerol was used as a carbon source at a concentration of 10% w/v.

The effect of reducing the sulphur content was also investigated. M. pulcherrima was cultured on sulphate concentrations of 50%, a 25% and 15% of the amount present in the basic media.

The final variable examined was pH which was adjusted using dilute HCl or KOH to produce the range pH 3-6. Each set of experiments lasted for 15 days and was tested for absorbance (O.D._{600 nm}) cell number and lipid fluorescence every 3 days.

### Sugar Sources

While M. pulcherrima can be grown on glycerol, potentially sourced from the biodiesel process, a far more abundant feedstock is lignocellulose. Waste food, agricultural wastes or energy crops grown specifically for the purpose can be converted into a range of sugars through relatively inexpensive chemical or enzymatic techniques. The composition of the sugars produced depends heavily on the method and source of the feedstock: however, the main sugars produced are glucose, arabinose, xylose and cellobiose, as well as a range of oligosaccharides.

M. pulcherrima has previously been shown to grow on a variety of sugars, aside from glycerol. These include glucose, galactose, L-sorbose, sucrose, maltose, cellobiose, trehalose, melezitose, D-xylose, N-acetyl-dglucosamine, ethanol, D-mannitol, D-glucitol, α-methyl-d-glucose, salicin, D-gluconate, succinate, and even alkanes such as hexadecane. The species can assimilate various nitrogen sources including ammonium, cadaverine, l-lysine and ethylamine.

To examine the ability of M. pulcherrima to grow on different sugar sources, 200 μl of culture were made up in 96-well plates in the standard media supplemented with a total concentration of either 15 g/L or 30 g/L of sugar for any one experiment.

After 72 h at 25° C., 180 rpm, the cultures were analysed for absorbance at 600 nm using a plate reader. All the possible combinations of two sugars were considered and six repeats for each combination were tested. The sugars examined were glucose, glycerol, xylose, arabinose, cellobiose, lactose, sucrose and glycerol.

The biomass productivity of the culture was determined by measuring O.D._{600 nm}.

### Waste Water Cultivation

To examine the ability of M. pulcherrima to grow on low cost media, M. pulcherrima was cultured at pH 5 in a minimum media without yeast extract. The composition of the optimised media was: KH₂PO₄ 7 g/L; Na₂HPO₄ 2.5 g/L, MgSO₄.7H₂O 0.188 g/L; MgCl₂.6H₂O 1.083 g/L; CaCl₂.2H₂O 0.15 g/L; ZnSO₄.7H₂O 0.02 g/L; (NH₄)₂SO₄ 0.063 g/L; NH₄Cl 0.405 g/L, glycerol 90 g/L. The medium was not sterilised prior to use. The cultures were maintained at 25° C. for 3 days, 180 rpm, before the temperature was changed to 15° C. and the agitation to 30 rpm. The same experimental conditions were then applied to using waste water with an additional 90 g/L of glycerol, as the medium. Both cultures were grown for 15 days and were analysed for the absorbance (O.D._{600 nm}) and lipid fluorescence every 3 days.

### Raceway Pond Cultivation

Two cultures were grown in the optimised minimum media given in section 2.4 with a reduced glycerol content of 30 g/L. The cultures were grown in two adjacent raceway ponds. Both ponds contained 500 L of culture and were situated in a climate controlled glasshouse. The ponds were inoculated with 500 ml of a M. pulcherrima culture (cultured over 48 hours) in a medium containing yeast extract 30 g/L, mannitol 5 g/L and sorbose 5 g/L at 25° C., and agitated at 180 rpm.

The cultures were agitated by a paddle wheel (10 rpm) and aerated through two spargers situated on opposite sides of the ponds. The cultures were checked for temperature, pH and absorbance at 600 nm until the beginning of the stationary phase, then every 4 days together with lipid fluorescence up to 28 days. With the onset of the stationary phase the temperature in the greenhouse was shifted from 25° C. to 20° C., the aeration was stopped and the paddle wheels were set at the minimum rotating rate.

### Lipid Fluorescence Analysis

The accumulation of lipid within M. pulcherrima cells was quantified using BODIPY staining. Bodipy is a green lipophilic dye with fluorescence on excitation with blue light at 493 nm proportional to lipid content.

The lipid fluorescence was measured using a Guava EasyCyte, Millipore flowcytometer and the data were analysed through a suitable software programme (Guavasoft, 2.2.2). Prior to the analysis, the cells were diluted up to an absorbance of around 0.2 (600 nm), then 100 μl of diluted culture were mixed gently with 5 μl of Bodipy dye and the volume was made up to 1 ml with distilled water. The samples were held in the dark for 30 min, before being exposed to light to halt the staining reaction. Subsequently, the solutions were diluted with distilled water (1:3) and analysed.

### Lipid Analysis

The lipid content was calculated gravimetrically. The quantification of the sterols was achieved by comparison of the integral of the peaks relating to the α-protons adjacent to the alcohol group of the sterol in the ¹H NMR and comparing this to the integral of the glyceride protons of the triglyceride backbone. FAME profiles were calculated by GC-MS calibrated to known standards. The GC-MS analysis was carried out using an Agilent 7890A Gas Chromatograph equipped with a capillary column (60 m×0.250 mm internal diameter) coated with DB-23 ([50%-cyanpropyl]-methylpolysiloxane) stationary phase (0.25 μm film thickness) and a He mobile phase (flow rate: 1.2 ml/min) coupled with an Agilent 5975C inert MSD with Triple Axis Detector. The FAME samples were initially dissolved in 2 ml of dioxane and 1 μl of this solution was loaded onto the column, pre-heated to 150° C. This temperature was held for 5 minutes and then heated to 250° C. at a rate of 4° C./min and then held for 2 minutes.

### Results

### Effect of the Nutrients on the Lipid Profile and Biomass Productivity

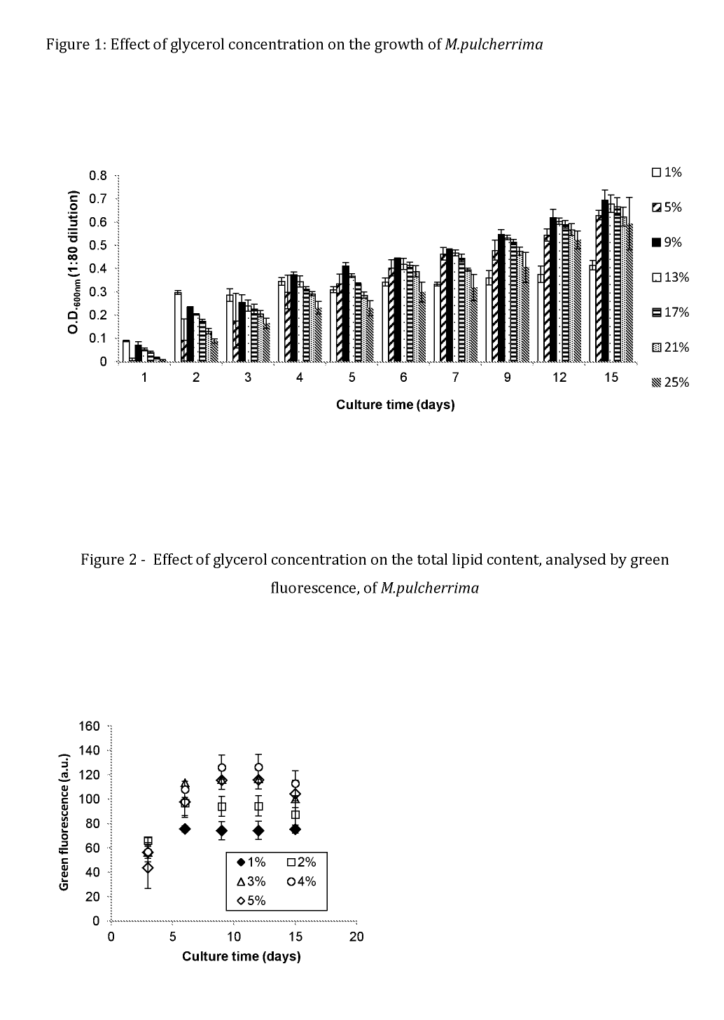

M. pulcherrima can be grown on glycerol, an important waste product of the biodiesel process (FIG. 1).

The biomass content, measured by O.D._{600 nm}, increased steadily over the whole 15 days, irrespective of the glycerol concentration.

The maximum biomass yield was observed with a 9% solution, after which a slight decrease in the biomass content was observed. This could be due to an excessive increase in the density which can have an effect on the oxygen uptake or simply that a high glycerol concentration stresses the yeast. The increase in biomass is only increased fractionally from using 5% glycerol to 9%.

The lipid for these samples was examined by staining with BODIPY, a green lipophilic fluorescent dye, and examining the absorbance on excitation with blue light at 493 nm. The fluorescence then gives a quantifiable measure of the total lipids from the sample analysed.

While the maximum concentration of biomass was achieved at a glycerol concentration of 9% the step change from using 5% is only very slight. This presumably is not a large enough increase in mass to justify almost doubling the glycerol concentration.

Starving the organism promotes lipid accumulation and energy storage in lipids, rather than further cell growth. To achieve this, the original nitrogen source added to the culture was in a limiting concentration. As the nitrogen was consumed before the carbon this allowed starvation to occur after this point. Lipids started to be accumulated after 3 days and reached a maximum after 9 days (FIG. 2). Lipids began accumulating 3 days after N depletion and reached a maximum after a further 9 days (FIG. 2).

A slight reduction was observed on culturing over 15 days where presumably the organism starts to use this storage for further growth.

The maximum lipid yield for M. pulcherrima was found when cultured on glycerol concentrations of 3-5 wt %.

A culture containing 3 wt % glycerol accumulated 5 g litre of biomass over 9 days (table 1). This biomass contained approximately 40% oil measured as dry weight.

The oil produced from M. pulcherrima was high, near 40% of the total dry weight and was found to be a mix of sterols and triglycerides.

The triglyceride portion has a relatively simple profile, rich in palmitic, palmitoleic, oleic and linoleic acids, while two sterols were isolated.

While the viscosity may be too high to use as a direct replacement for diesel oil, the bio-oil produced is an appropriate viscosity for use in care products. The energy density is higher than ethanol, presumably due to the lower oxygen content and an alternative use for this interesting feedstock is through further chemical upgrading by decarboxylation or through inter-esterification to a suitable biofuel.

TABLE 1
Biomass and oil characteristics recovered from 500 l tank reactor

| Content | | Total |
|---|---|---|
| Biomass | | 0.1-22 g L⁻¹ |
| Oil content | | Up to 55 wt % dry weight |
| Sterols | | 0-50% (total lipid) |
| Glyceride lipid | | 50-100% |
| | 14:0 | 0.1-5% |
| | 15:0 | 0.1-5% |
| | 16:0 | 5-25% |
| | 16:1 | 0.1-20% |
| | 17:0 | 0.1-5% |
| | 18:0 | 0.1-10% |
| | 18:1 | 30-70% |
| | 18:2 | 5-25% |
| | 18:3 | 0.1-25% |
| | 19:0 | 0.1-5% |
| | 21:0 | 0.1-5% |
| Density | | 0.76-1.1 g L₋₁ |
| Dynamic viscosity | | 0.0058-0.58 cP |
| Energy density | | 27-40 MJ L₋₁ |

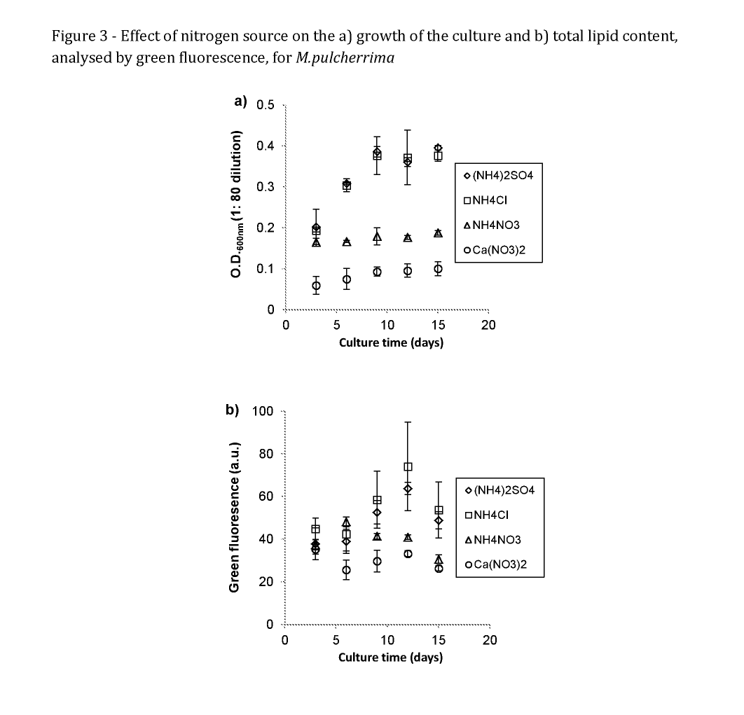

One key issue with using wastes are a feedstock is the heterogeneity of the supply. To produce an organism that can be successfully cultivated in wastes, a degree of flexibility is required. To further assess M. pulcherrima for its potential to use these feedstocks the effect of the nutrients on the growth and lipid content M. pulcherrima was assessed. A range of nitrogen sources were examined (FIG. 3). The biomass productivity was found to be far higher when cultivated with ammonium salts compared to pure nitrates. This was also the case for the total lipid content of these cultures, though the discrepancy is smaller.

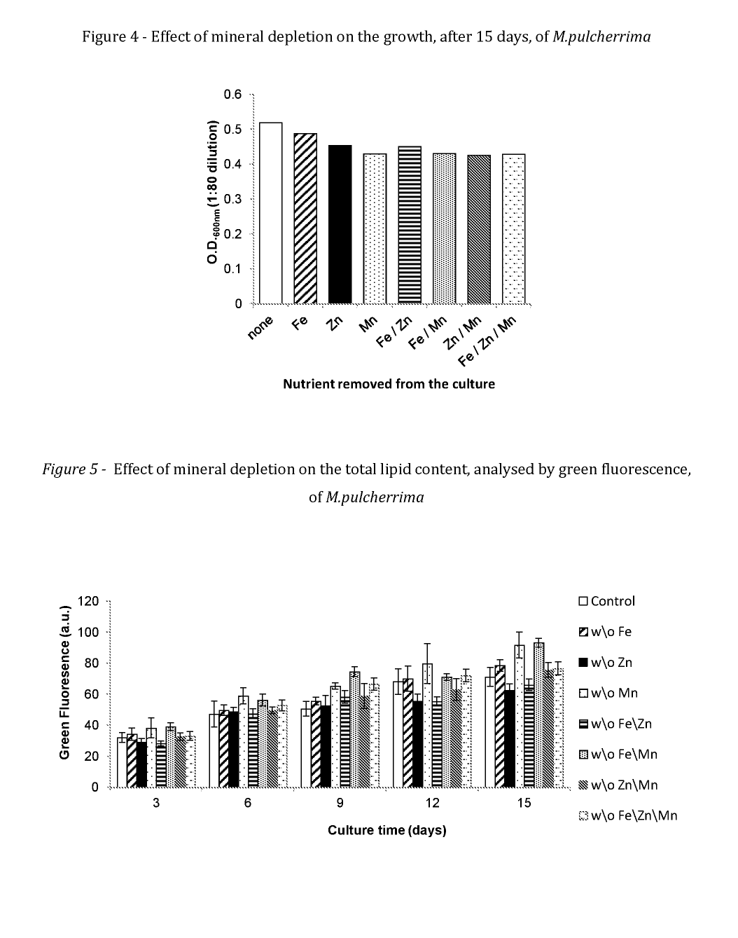

A range of micronutrients are added to most yeast cultures, these include salts of manganese, zinc and iron. All of these micronutrients are present in waste water, though the type of salt and the amount is highly dependent on the source and season. To determine the flexibility of M. pulcherrima to potential changes in the culture media, the yeast was grown in a range of nutrient deficient cultures (FIG. 4). While the highest growth was observed with all the nutrients present, only a 15% reduction in biomass when removing all iron, zinc and manganese from the culture.

The lipid productivity was also not significantly affected by the removal of nutrients. The highest lipid amounts were achieved when all manganese was removed, even above that of the control. However, there was a 10% reduction in the lipid content, compared to the control in all conditions examined where zinc was removed.

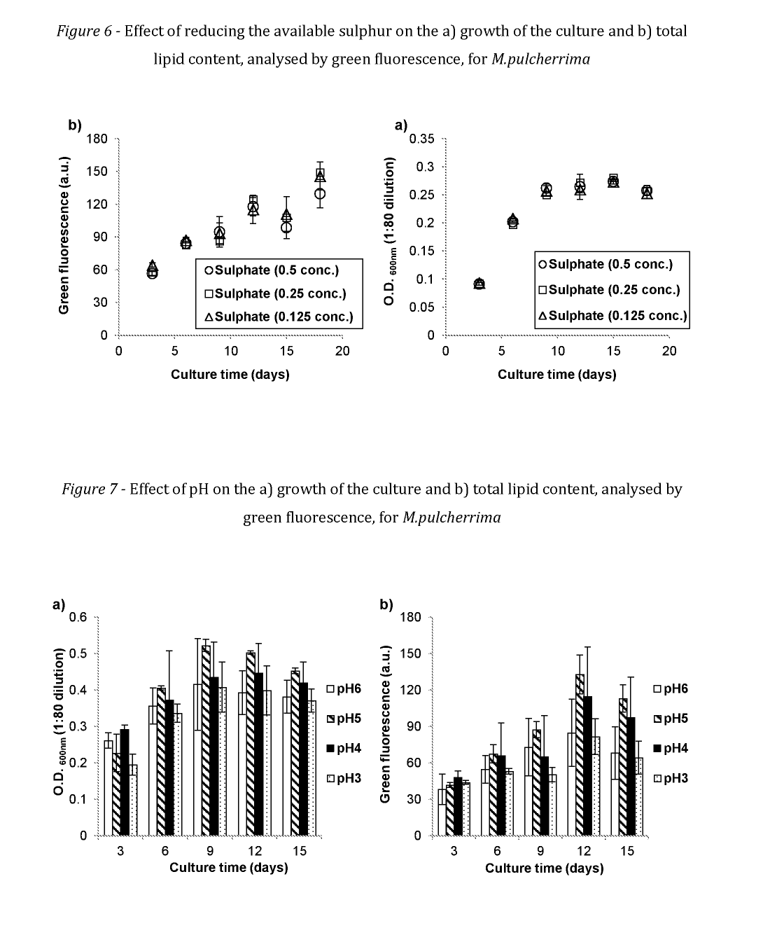

Another key nutrient that can affect the growth rate is sulphur. Generally sulphur is present as sulphates, especially in waste water. To assess the flexibility of M. pulcherrima, a range of cultures were examined with reduced levels of sulphate (FIG. 6). There is no difference of culturing M. pulcherrima in low or high sulphate conditions, the same level of biomass and high lipid levels are obtained.

M. pulcherrima demonstrates good flexibility on being cultured with different types and amounts of nitrogen, sulphur and micronutrients. This demonstrates that while there is an optimal culture that produces the highest levels of lipids from any given system, the effect of removing key nutrients does not dramatically reduce lipid yields. M. pulcherrima therefore has excellent adaptability to any changes in the waste water or alternative nutrient streams that could potentially be used to culture the system.

### Environmental Factors

One of the factors in maintaining a monoculture is the ability of M. pulcherrima to be cultured at low pH. In the literature M. pulcherrima has been reported to grow optimally at pH between 5 and 7.5, though has been shown to grow at a pH as low as 3. M. pulcherrima also regulates this environment and will change the pH up or down depending on the stage of the lifecycle. Under the conditions used in this study M. pulcherrima can grow under a variety of pH levels, though the maximum biomass was observed at pH 5 (FIG. 7). This was reduced slightly at pH 4. Surprisingly, even at pH 3 around 85% of the biomass was produced compared to that at pH 5. Irrespective of the pH, or change in the pH of a culture, high levels of biomass are still produced. Lipid accumulation is more heavily affected by a change in the pH however. The maximum lipid observed was found when the initial pH values were between pH 4 and 5 and there was a significant reduction in the cultures held at pH 3 or 6. While the more acidic conditions could potentially make a large difference to the threat of invasion, if the pH is too low, then the lipid yield will be reduced substantially.

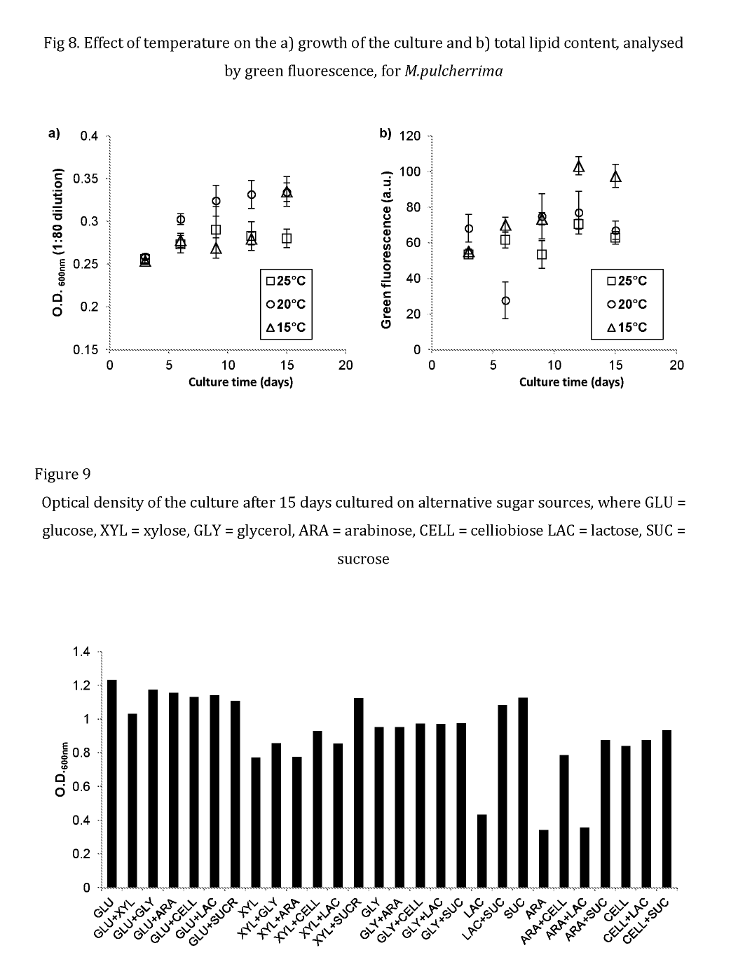

A further trait to deter invasive organisms is to culture M. pulcherrima at low temperatures (FIG. 8). To examine the effect of temperature on the yeast, M. pulcherrima was cultured at 25° C. for 3 days, the temperature was then modified for the remaining length of the culture. M. pulcherrima grows extremely well between 15° C. and 20° C., not only are these temperatures too low for most common bacteria but are ideal for producing fuels in Northern Europe. At 25° C. a 20% reduction in biomass is observed, though there is little difference in the lipid production of the system to that produced at 20° C. The highest lipid productivity was observed at 15° C. As low temperatures are reported to be a triggering factor in the sporulation process and the pulcherrima cells are a transition state leading to spores, this explains the ability at this point to increase in lipid content.

### Alternative Feedstocks

While M. pulcherrima can be grown on glycerol, potentially sourced from the biodiesel process, a far more abundant feedstock is lignocellulose. Waste food, agricultural wastes or energy crops grown specifically for the purpose can be converted into a range of sugars through relatively inexpensive chemical or enzymatic techniques. The composition of the sugars produced depends heavily on the method and source of the feedstock, however, the main sugars produced are glucose, arabinose, xylose and cellobiose, as well as a range of oligosaccharides. To this end M. pulcherrima was cultivated on a range of these sugars including lactose and sucrose to mimic further oligosaccharides that could potentially be produced from these abundant sources (FIG. 9).

M. pulcherrima grows better on glucose than on any other sugar or combination of sugars. When glucose is present any other sugar can be used as a carbon source with little reduction in the biomass yield. While M. pulcherrima is capable of being cultured on xylose the biomass productivity is reduced somewhat. M. pulcherrima struggles to grow on the arabinose aldehyde, and lactose but metabolises sucrose extremely effectively. Overall, M. pulcherrima can be cultivated effectively on a range of sugars, especially if glucose is also present. M. pulcherrima would be able to metabolise a large number of waste products carbon sources derived from waste streams.

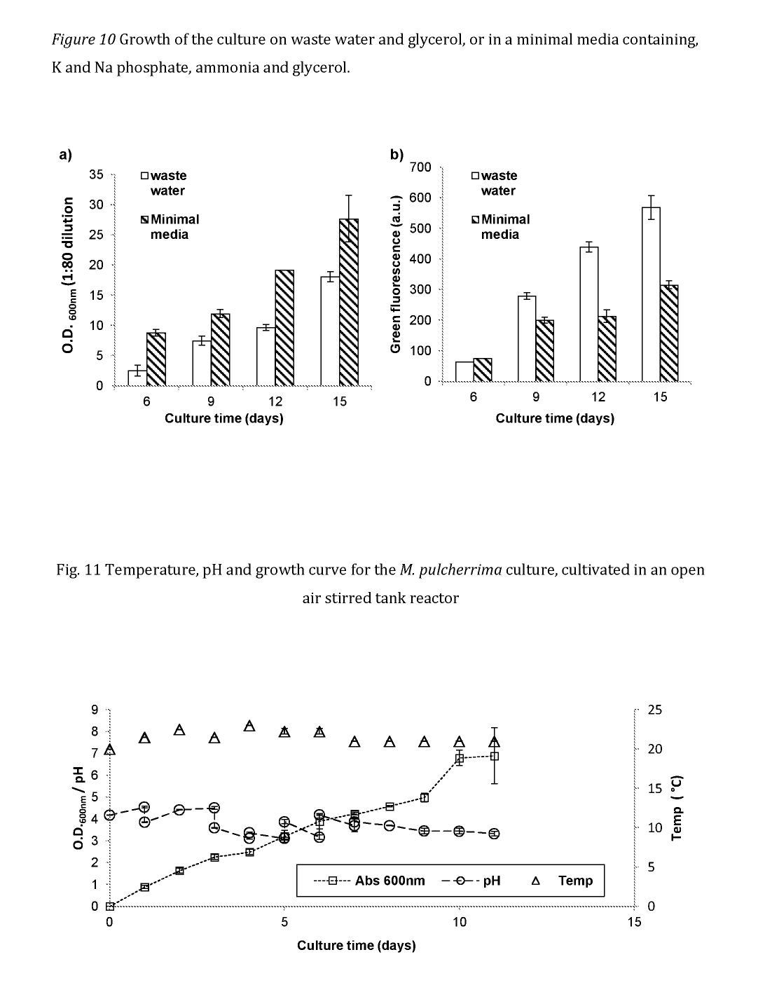

A further consideration in producing biofuels is the source of phosphorous and nitrogen. A minimal media, made up without yeast extract, was used to mimic this and compared to actual waste water in the cultivation of M. pulcherrima with glycerol (FIG. 10).

The minimal media is perfectly adequate for growth, large densities of M. pulcherrima were extracted (5 g/L biomass) over the 15 days. Waste water was not as effective in producing biomass. However, the minimal media was not as conducive in producing total lipids. This is presumably because of a difference in the C/N ratio between the two conditions meant that the formation of pulcherrima cells was quicker under the waste water conditions producing less biomass but more lipid. This demonstrates that feedstocks available on a large enough scale for fuel production are ideally suited to culturing M. pulcherrima for lipids.

### Stirred Tank Production

To assess the ability of M. pulcherrima to be cultured in truly non-sterile conditions in open tanks, two 500 L raceway ponds were used (FIG. 11).

In these the yeast was grown on the minimal media. However, to induce further lipid production the glycerol content was reduced to 30 g/L. The ponds were situated in a temperature controlled greenhouse and the solution was gently agitated by the paddle wheel at 10 rpm. The culture was held for 28 days.

Two cultures were initiated in the modified Chatzifragkou medium with a glycerol content of 30 g/L. The cultures were grown in two adjacent raceway ponds. Both ponds contained 500 L of culture and were situated in a climate-controlled glasshouse. The ponds were inoculated with 500 ml of a M. pulcherrima grown for 48 hours in a YMS medium containing, yeast extract 30 g/L, mannitol 5 g/L and sorbose 5 g/L at 25° C., 180 rpm.

The cultures were agitated by a paddle wheel (10 rpm) and aerated with air supplied using an air-flow pump through two spargers situated on opposite sides of the ponds. The cultures were monitored for temperature, pH and absorbance at 600 nm until the beginning of the stationary phase, then every 4 days together with lipid fluorescence up to 28 days. With the onset of the stationary phase the temperature in the greenhouse was shifted from 25° C. to 20° C., the aeration was stopped and the paddle wheels were set at the minimum rotating rate. Biomass productivity, temperature and pH were recorded over a 10-day period (FIG. 11).

Over the course of the culture the biomass productivity was seen to increase steadily. The temperature remained roughly constant irrespective of the conditions outside of the greenhouse at 21° C. During the course of the culture M. pulcherrima regulates the environment by producing both acids and bases throughout depending on the stage of the growth cycle. To maintain a healthy population of M. pulcherrima, while retaining reasonable lipid and biomass concentrations the pH was artificially kept between 3 and 4 through the addition of weak solutions of either HCl or KOH. While some bacteria was observed at various points over the first 72 hours of culture, the population remained overwhelmingly M. pulcherrima, after the pH had continually been near 3 no contamination was observed from this point on. Under high biomass conditions many colonies were observed sticking to the paddle wheels. These colonies developed an intense pink colour. The colour is due to the production of pulcherrimin, presumably produced by these cultures and less so in the submerged biomass due to the abundance of oxygenation.

The productivity was found to be 1.25 g/L biomass. This biomass had a lipid content of 35%,

Oleaginous Biomass Production by M. pulcherrima in a Two-Step Process Controlled by Nitrogen Starvation and Low pH.

The growth of M. pulcherrima biomass containing high levels of accumulated lipid was achieved using a novel two step process:

Step 1—production of actively growing yeast cell culture that contained low levels of accumulated oil (0-10% w/v);
Step 2—triggering the culture to produce a high proportion (40-50%) of oil-rich (40-80% w/v) pulcherrima cells. Crucially the method blocks pulcherrima cells forming spores that are very low in accumulated oil (0-5% w/v).

The method involved controlling two factors: nitrogen availability and pH. The method resulted in a dramatic increase in oil productivity of the M. pulcherrima cultures.

### Step 1:

Pre-inoculation cultures were grown from a single colony of M. pulcherrima taken from a YMD agar plate, dissolved in 10 ml YMD (yeast extract 10 g/L; malt extract 20 g/L; glucose 20 g/L). Pre-inoculation cultures were used to inoculate 10 ml of modified Chatzifragkou medium (Chatzifragkou et al 2010) in 50 ml falcon tubes.

Modified Chatzifragkou medium consisted of: KH₂PO₄ 7 g/L; Na₂HPO₄ 2.5 g/L, MgSO₄.7H₂O 1.5 g/L; CaCl₂.2H₂O 0.15 g/L; ZnSO₄.7H₂O 0.02 g/L; MnSO4.H₂O 0.06 g/L, FeCl₃ 0.15 g/L; (NH₄)₂SO₄ 0.5 g/L and yeast extract 1 g/L.

All media was autoclaved for 2 hours at 120° C. prior to use. The cultures were maintained at 25° C. with an agitation rate of 180 rpm.

Cultures were grown in minimal medium supplemented 1, 5, 9, 13, 17, 21, 25% glycerol and the biomass content measured over a 15 day period by O.D._{600 nm}. (FIG. 1). The maximum biomass yield obtained was 28 O.D._{600 nm} units (approximately 5 g/L dry weight) with a 9% solution of glycerol in minimal medium, but higher (13, 17, 21, 25%) and lower (5%) concentrations were also effective.

### Step 2:

To trigger high levels of lipid production by the culture two variables were manipulated—nitrogen availability and pH.

Nitrogen Availability without pH Control

The nitrogen source ((NH₄)₂SO₄, NH₄Cl, NH₄NO₃ and Ca(NO₃)₂) was present in the culture medium at a limiting concentration of 0.2 g/L. The nitrogen source was depleted progressively from day 3 of the culture, reaching very low levels (0.084 g/L) by day 15 before the carbon source (glycerol or alternative).

pH in these cultures was not artificially controlled in these cultures, but measurements showed that the culture spontaneously lowered the pH to between 2.0 and 3.5.

Nitrogen Availability with pH Control

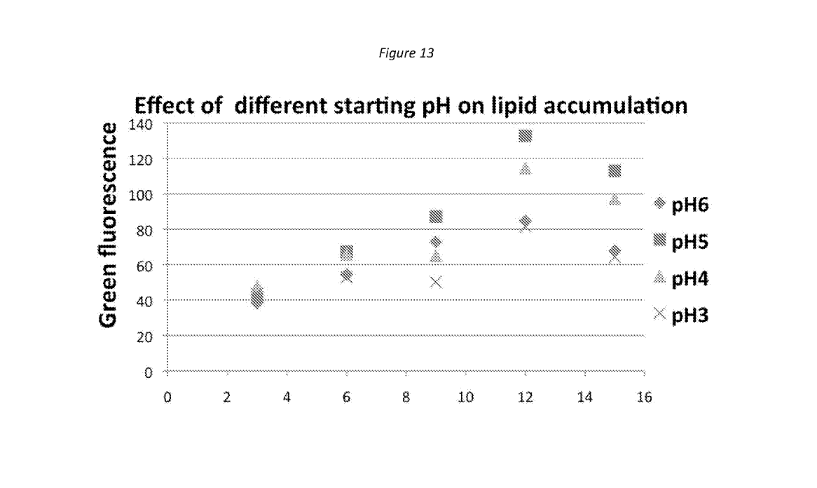

A series of cultures at different starting pHs were set up as described for Step 1 and lipid accumulation quantified at day 15 by Bodipy staining. The data in FIG. 13 shows that maintaining a starting pH of 5 maximized the oil productivity of M. pulcherrima cultures triggered to accumulate lipid by nitrogen starvation.

Culture of M. pulcherrima at Low Temperature Further Increases Oil Production in the Two-Step Process

To examine the effect of temperature on biomass production and oil accumulation M. pulcherrima was cultured at 25° C. for 3 days in modified Chatzifragkou medium. The temperature was then reduced for the remaining length of the culture (to day 15). Highest biomass production was achieved between 15° C. and 20° C. (FIG. 3) The highest lipid productivity as measured by Bodipy fluorescence was achieved at 15° C. Low temperature is reported to trigger the sporulation process in M. pulcherrima. If sporulation is blocked by low pH, low temperature results in an increase in the proportion of oil-rich pulcherrima cells in the biomass.

M. pulcherrima Produces High Levels of Oil when Cultured on Diverse Nitrogen Sources

M. pulcherrima was cultured on a range of nitrogen sources (NH₄Cl, NH₄NO₃ and Ca(NO₃)₂) by replacing (NH₄)₂SO₄ in the medium described in Example 1a. These reagents were added in precise concentrations to achieve a consistent nitrogen quantity (N=0.2 g/L). Glycerol was used as a carbon source at a concentration of 10% w/v. using the conditions described in Example 1a above. The biomass productivity was found to be higher (ammonium OD=>20; nitrate OD=<1.0, when cultivated with ammonium salts compared to pure nitrates (FIG. 4). This was also the case for the total lipid content of these cultures, though the difference was smaller.

### Further Example—Two Step Process

To achieve high oil production by Metschnikowia pulcherrima a two step process was applied.

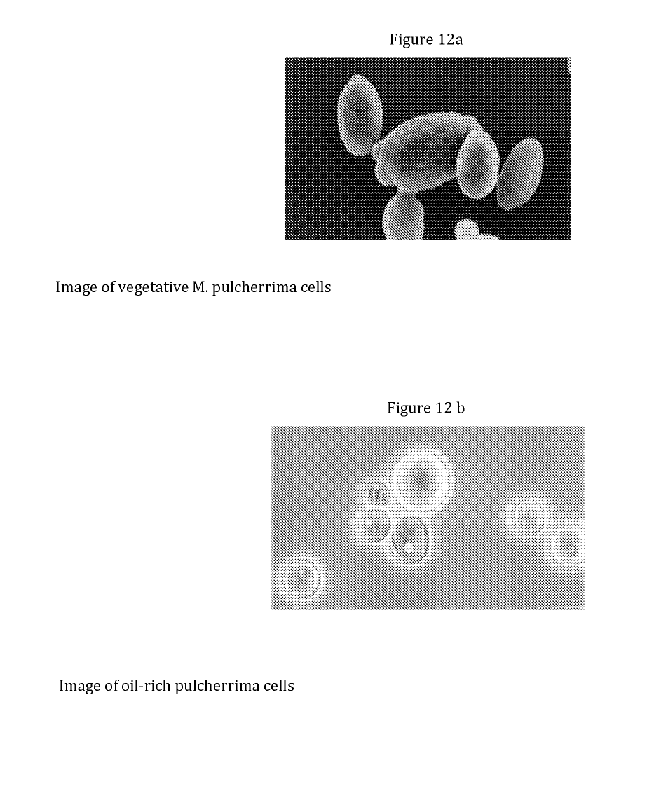

Step 1: Purpose: produce large amounts of non-oleaginous biomass composed of oval-shaped vegetative pulcherrima cells (FIG. 12a).

This phase is sustained by relatively high nutrient levels (minimal medium)—carbon source such as glycerol; nitrogen sources such as ammonia, yeast extract; sulphur source such as magnesium sulphate and/or ammonium sulphate; and a pH maintained between 4.0-4.5.

M. pulcherrima may naturally lower pH to 2.0-3.0; therefore it may be necessary to monitor and adjust pH by adding a base e.g. NaOH to raise pH to the optimum. Occasionally the culture may become too basic, in which case pH can be lowered by addition of HCl. The optimum temperature. for vegetative growth is 20-25° C.

Step 2: Purpose: promote production of oil-rich pulcherrima cells (FIG. 12b) and block their progression to spores.

The production of oil-rich pulcherrima cells from vegetative M. pulcherrima cells is triggered by starvation for either nitrogen and/or sulphur (or for other nutrients and microelements.

The vitamin biotin can also be provided to trigger pulcherrima cell production.

Nitrogen and sulphur are gradually and naturally depleted from the growth medium by growth of vegetative M. pulcherrima cells until their level(s) become too low to sustain normal growth.

One consideration is the ratio of carbon to nitrogen or sulphur. The most effective starting ratio is 60 C:1N (300 C:1S). At the end of culture, the amount of available nitrogen has depleted to a maximum of 0 g/L.

The culture medium contains deliberately limiting amounts of nitrogen and sulphur (nitrogen=0.2 g/L/sulphur 0.04 g/L) to ensure that starvation begins after 3-4 days of culture. The vegetative cells then begin the process of sporulation to form needle-shaped oil-poor spores (FIG. Z). Significantly for this method, the vegetative M. pulcherrima cells first form a transition cell type, the oil-rich pulcherrima cell. The formation of oil-rich pulcherrima cells from vegetative M. pulcherrima cells is enhanced by maintaining the culture at a temperature below 20° C. but not less than 10° C.

Progression from oil-rich pulcherrima cells to oil-poor spores is efficiently blocked by lowering the culture to pH 2.5-3.5. This allows the culture to accumulate a high proportion of pulcherrima cells (40-80% of the cells; average>50%)

### Further Example—Fed-Batch Culture

Fed-batch allows greater biomass concentrations to accumulate prior to initiating oil production by starvation for nitrogen/sulphur. This allows more efficient production since smaller culture vessels are required to produce an amount of biomass compared to the simple two step process which is limited by the relatively low starting concentration of nutrients (nitrogen/sulphur) resulting in lower biomass concentrations.

In this example a 100 ml culture was inoculated with a 24 h pre-inoculum culture of M. pulcherrima. 4 different conditions were tested:
Condition 1: With yeast extract 0.1%
Condition 2: Without yeast extract and with 0.4 μg/L biotin
Condition 3: Without yeast extract and without biotin
Condition 4: Without yeast extract, with 0.4 μg/L biotin and with a supplement of NH₄Cl (final C:N ratio 20:1)
All the media initially contained the following nutrients:
7 g/L KH2PO4
2.5 g/L Na2HPO4
0.02 g/L ZnSO4*7H2O
0.188 g/L MgSO4*7H2O
1.083 g/L MgCl2*6H2O
0.063 g/L (NH4)2SO4
0.405 NH4Cl (0.068 in biotin or no biotin cultures, 1.273 g/L in cultures with C:N ratio 20:1)
0.15 g/L CaCl2*H2O
7.5 g/L Glucose
7.5 g/L Xylose
7.5 g/L Arabinose
7.5 g/L Cellobiose

After 3 days at 25 C, 180 rpm, the cultures were supplemented with all the nutrients except phosphate salts, in a quantity half the initial amount for the first 15 days, then increasing up to the same initial value. Phosphate salts were added only at 15 days in the same initial quantities present at initiation. The volume of feeding was kept between 5 and 10 ml each time.

The feeding was done at days 5, 7, 9, 11, 13, 15, 17, 19 and 21 from the inoculation.

### Measurements:

The following measurements were recorded: 600 nm, dry weight and pH value. The dry weight was measured using 2 ml samples from the cultures, which were centrifuged at 13000 rpm for 10 min, separated from their supernatants and placed at 75 C for 24-48 h. The pH was kept at approximately 5 in order to maximize the growth rate.

After 24 days, the pH was lowered to 3 and the cultures were maintained without agitation at 15 C for 2 weeks without further feeding. After 14 days, the cultures were analysed for absorbance at 600 nm and pH and then centrifuged to recover the biomass for dry weight calculations and lipid analysis.

The fed batch cultures produced the following amounts of dry biomass:

Condition 1 (with yeast extract 0.1%): 17.3 g\L+\−1.5
Condition 2 (without yeast extract and with 0.4 μg/L biotin): 14.3 g\L+\−0.3
Condition 3 (without yeast extract and without biotin): 13.4 g\L+\−0.9
Condition 4 (without yeast extract, with 0.4 μg/L biotin and with a supplement of NH₄Cl (final C:N ratio 20:1)): 14.6 g\L+\−1.1

### Dewatering by Self-Flocculation

A 900 L culture of the yeast (28 days old; nitrogen starved; at a cell density of approx. 8 g/L wet biomass) was transferred into a 1000 L capacity Conical Biofuel Tank (Smiths of the Forest of Dean Ltd, The Orchard, Station Road, Milkwall, Coleford, Gloucestershire GL16 8PZ) using a peristaltic pump. The culture was maintained at approximately 20° C. for 2 weeks and then at 15° C. for 2 more weeks, although higher or lower temperatures are functional. Approximately 90% of the yeast biomass settled to the bottom of the tank by self-flocculation over an approximately 24 hour period. The biomass remaining in solution can be precipitated by adding a flocculation agent such as alginate using standard methods commonly used in the wine making industry, for example.

The concentrated biomass was transferred from the tank to suitable containers using a tap at the base of the tank. Further dewatering was achieved using centrifugation (8 rpm/g for 10 mins) that resulted in a paste suitable for oil extraction by methods described in Example 3.

Extraction of Oil from M. pulcherrima Biomass

Oil may be easily extracted from the biomass by known methods. Methods appropriate for extraction of the oil from the biomass are for example, the solvent method or microwave extraction.

Using the solvent method, the yeast (either freeze dried or still containing up to 95% water) was suspended in a large excess of solvent (from 0.1 g-500 g biomass in 0.1-5 L of solvent) with stirring, the solvents used were dichloromethane, chloroform, chloroform and methanol, hexane or diethyl ether (or any combination). The yeast was stirred at room temperature or anywhere up to the reflux temperature of the solvent. This process was undertaken for between 30 minutes to 72 hours, depending on the size of the sample and the temperature used.

Alternatively soxhlet equipment was also used, using the same solvents and conditions. In a typical soxhlet extraction, 0.1 g of microbial biomass was added to a cellulose finger in Soxhlet glassware and the lipids extracted over 0.5, 1, 2, 4, 12, 24 or 48 hours with a 2:1 CHCl₃/MeOH mixture (50 ml).

An alternative method was to use a microwave extractor. Typically an Anton Parr monowave 300 microwave reactor was used equipped with a MAS 24 autosampler capable of loading 10 ml sealable reaction vessels (capable of sustaining a pressure of 30 bar). In a typical experiment the yeast biomass, either freeze dried or containing up to 95% water (0.1 g) was suspended in a 2:1 CHCl₃/MeOH mixture (6 ml) with a stirrer bar. The microwave was set on an automated cycle containing 1) Heating to the desired temperature and pressure (typically taking less than 1 minute) with 1000 rpm stirring, 2) the reaction (0.5-20 minutes, 1000 rpm stirring) 3) fast cooling using compressed N₂ (typically less than 2 minutes depending on temperature). The resulting oil was extracted into chloroform/methanol and washed with water three times, the organic solvents was then removed under reduced pressure prior to the analysis.

### Further Processing/Refining

Once the oil has been extracted further chemical processing can be undertaken. Two methods to produce fuels are described herein, a though the oil extracted can be used directly, without further processing.

The oil produced from M. pulcherrima containing any combination of sterols, neutral or polar lipids can be passed into a reaction zone, between 200-500° C. comprising of a catalyst in the presence of either hydrogen or an inert gas. Through the deoxygenation, hydrogenation, isomerization or cracking (or any combination of the four) of the feedstock a paraffin rich stream (the paraffins will contain from about 4 to about 30 carbon atoms) will be produced in addition to carbon dioxide and water.

The oil produced from M. pulcherrima can be passed into a reaction zone, between 20° C.-350° C. comprising of no, one or combination of catalysts, in the presence of either an alcohol, acid anhydride, organic acid or ester (or any combination). Through the esterification of the sterol and lipid feedstock an oil containing the original starting products, glyceryl esters, sterol esters and fatty acid esters will be produced.

1. A method of increasing lipid accumulation in pulcherrima cells by culturing a yeast in a culture medium under conditions suitable for promoting production of pulcherrima cells and inhibiting sporulation.

2. The method as claimed in claim 1, wherein the method comprises a first step of culturing the yeast under conditions suitable for promoting production of vegetative pulcherimma cells, and a second step of culturing the yeast under conditions suitable for inhibiting sporulation.

3. The method as claimed in claim 2, wherein the first step comprises providing the yeast with at least one nitrogen and/or sulphur source, and at least one carbon source.

4. The method as claimed in claim 3, wherein the nitrogen and/or sulphur source is provided in the culture medium in a limiting concentration to induce starvation of the yeast.

5. The method as claimed in claim 4, wherein the least one nitrogen source is provided at the limiting concentration of about 0.15 to about 1.4 g/L.

6. The method as claimed in claim 4, wherein the least one sulphur source is provided in a the limiting concentration of about 0.04 g/L or lower.

7. The method as claimed in claim 3, wherein the amount of available carbon is 12 g/L or higher.

8. The method as claimed in claim 2, wherein the first step comprises maintaining a temperature of about 10 to about 28° C.

9. The method as claimed in claim 8, wherein the first step comprises maintaining a temperature of about 20 to about 25° C.

10. The method as claimed in claim 2, wherein the first step comprises maintaining the pH at about 4 to about 6.

11. The method as claimed in claim 2, wherein the second step comprises lowering the temperature.

12. The method as claimed in claim 11, wherein the step of lowering the temperature comprises adjusting the temperature to below about 20° C.

13. The method as claimed in claim 2, wherein the second step comprises adjusting the pH to between about 2 to about 4.

14. The method as claimed in claim 2, wherein the first step comprises providing the yeast with biotin.

15. The method as claimed in claim 1, wherein the yeast is selected from:
Metschnikowia pulcherrima, Metschnikowia fructicola, Metschnikowia reukaufi, Candida albicans, Chlamydozyma zygote, Metschnikowia vanudenii, Metschnikowia lachancei, Hansenula saturnus and Debaryomyces dekkeri.

16. The method as claimed in claim 1, comprising the step of obtaining oleaginous biomass from the culture medium.

17. The method as claimed in claim 16, wherein the oleaginous biomass comprises lipid at about 40% of total dry weight.

18. The method as claimed in claim 17, wherein the lipid comprises sterols, triglycerides and/or free fatty acids.

19. The method as claimed in claim 18, wherein the triglycerides comprise palmitic acid, palmitoleic acid, stearic acid, oleic acid and/or linoleic acid.

20. The method as claimed in claim 2, wherein the at least one carbon source is selected from glycerol, lignocellulose, sugar, waste water, waste foods, agricultural waste or energy crops.

21. The method as claimed in claim 20, wherein the at least one carbon source comprises glucose.

22. The method as claimed in claim 20, wherein the at least one carbon source comprises glycerol added to the culture medium at a concentration of 3 to 5 wt %.

23. The method as claimed in claim 2, wherein the at least one nitrogen source comprises ammonium salts.

24. The method as claimed in claim 1, wherein the culture medium comprises nutrients selected from salts of manganese, zinc, sodium potassium, calcium, magnesium and iron.

25. The method as claimed in claim 1, wherein the culture medium is unsterilized culture medium.

26. The method as claimed in claim 1, wherein the yeast is cultured in a substantially open reactor.

27. The method as claimed in claim 26, wherein the reactor comprises an open raceway pond.

28. The method as claimed in claim 1, further comprising the step of dewatering the oleaginous biomass.

29. The method as claimed in claim 28, wherein the step of dewatering the oleaginous biomass comprises a self-flocculation step.

30. The method as claimed in claim 28, wherein the step of dewatering the oleaginous biomass comprises a precipitation step.

31. The method as claimed in claim 1, further comprising the step of extracting lipid from the oleaginous biomass.

32. The method as claimed in claim 31, wherein the step of extracting lipid from the oleaginous biomass is by solvent or microwave extraction.

33. The method as claimed in claim 31, wherein the step of extracting lipid from the oleaginous biomass is performed between 3-15 days.

34. The method as claimed in claim 31, comprising at least one step of further chemical upgrading.

35. The method as claimed in claim 34, wherein the chemical upgrading is to produce a fuel, fuel substitute, base for cosmetics, plastic or animal feed.

36. The method as claimed in claim 1,
wherein the oleaginous biomass further comprises co-products.

37. The method as claimed in claim 36, wherein the co-products comprise pulcherrimin pigment, pulcherriminic acid, animal feed, ethyl caprylate, acetoin, isoamyl alcohol, 2,3 butanediol, acetic acid, acetaldehyde, n-propanol, 1,2-methyl-1-propanol, 2,3-butanediol, 2-phenylethanol, geranyl acetate, geranyl alcohol, ethyl acetate, ethyl hexanoate and/or ethyl decanote.

38. An oil comprising a lipid profile comprising 0-50% sterol, 50-100% glyceride and 0-10% free fatty acids.

39. The oil as claimed in claim 38, having a dynamic viscosity of about 0.58 Pa-s measured at 40° C.

40. The oil as claimed in claim 38, having an energy density of 27.33 MJ/kg.

41. The oil as claimed in claim 38, wherein the oil is a bio-oil.

42. A fuel, fuel substitute, base for cosmetics, animal feed or plastic comprising the oil of claim 38.

43. (canceled)

44. A yeast culture comprising pulcherimma cells at greater than about 0.1%(w/v).

45. The yeast culture as claimed in claim 44 comprising pulcherimma cells at greater than about 20% (w/v).

46. The yeast culture as claimed in claim 45, comprising pulcherimma cells at greater than about 40%(w/v).

47. A pulcherimma cell comprising lipid at about 25-80% (w/v).

48. (canceled)
